(19) United States
(12) Patent Application Publication
(10) Pub. No.: US 2023/0193350 A1
(43) Pub. Date: Jun. 22, 2023

(54) METHOD FOR DIAGNOSING AND TREATING BLOOD CANCER

(71) Applicants: Ohara Pharmaceutical Co., Ltd., Koka-shi Shiga (JP); Saga University, Saga-shi, Saga (JP)

(72) Inventors: Yuki Kurahashi, Koka-shi Shiga (JP); Magoichi Sako, Koka-shi Shiga (JP); Hiroo Wakita, Koka-shi Shiga (JP); Shinya Kimura, Saga-shi Saga (JP); Tatsuro Watanabe, Saga-shi Saga (JP); Yuta Yamamoto, Saga-shi Saga (JP)

(21) Appl. No.: 17/919,735

(22) PCT Filed: Jun. 11, 2021
(86) PCT No.: PCT/JP2021/022226

§ 371 Date: Oct. 18, 2022

(30) Foreign Application Priority Data
Jun. 15, 2020 (JP) 2020-102904

(51) Int. Cl. C12Q 1/42; A61K 31/53; A61K 31/497; A61P 35/00

(52) U.S. Cl. CPC C12Q 1/42; A61K 31/53; A61K 31/497; A61P 35/00; G01N 2800/7028

(57) ABSTRACT

The present invention provides: a method for identifying a blood cancer patient who may benefit from a monotherapy using a drug comprising a DNA methyl transferase inhibitor and a combination therapy using the aforesaid drug together with another epigenetic controller; a pharmaceutical composition to be used in treating a patient who has been identified or selected by the method; and a kit for identifying such a patient. More particularly, the aforesaid method comprises a step for measuring the expression amount of DUSP5 in a sample obtained from a blood cancer patient. When the expression amount measured in this step is lower than a reference expression amount of DUSP5, then the patient is identified or selected as a blood cancer patient who may benefit from a treatment using the drug comprising a DNA methyl transferase inhibitor.

### TECHNICAL FIELD

The present invention relates to a method for identifying a blood cancer patient who may benefit from a treatment using a drug comprising a DNA methyl transferase inhibitor, a pharmaceutical composition for use in treating a patient who has been identified by the method, and a kit for identifying the patient.

### BACKGROUND TECHNOLOGY

Dual-specificity phosphatase 5 (DUSP5) is a member of DUSP family. These phosphatases inactivate target kinase such as mitogen-activated protein kinase by dephosphorylating both phosphoserine/threonine and phosphotyrosine residues, which are substrate proteins. It has been reported that DUSP5 is localized in the nucleus of cells, particularly has dephosphorylation effect on ERK1/2 (non-patent document 1). DUSP5 gene is called as tumor suppressor gene, because it has the effect of inactivating ERK1/2, and gene expression of DUSP5 is suppressed in gastric cancer and rectal colon cancer (non-patent document 1). In addition, it has been reported that in gastric cancer, the DUSP5 expression is changed by DNA methylation of DUSP5 gene, and DUSP5 gene is controlled by epigenome (non-patent document 2).

Epigenome drugs are said to show anti-cancer effects by normalizing epigenetic abnormalities of cancer cells. To give an example of a DNA demethylation agent, following mechanism of the effect thereof has been reported (non-patent document 3). In cancer cells, the expression of tumor suppressor gene is suppressed by methylation of cytosine guanine dinucleotide (CpG) in the region involved in the expression of tumor suppressor gene. However, by administering a DNA demethylating agent, CpG is demethylated. As a result, the suppression on the expression of the tumor suppressor gene is released, and the proliferation of cancer is suppressed. Regarding other epigenome drugs, although the target epigenome abnormalities are different, they are common in that the suppression of gene expression is released and the gene expression is recovered (non-patent document 3). However, it is not clear that expression recovery of which gene is important for anticancer effects. Therefore, biomarkers are needed to predict the therapeutic effect of an epigenome controller.

### PRIOR ART DOCUMENTS

### Non-Patent Documents

1. Chen H-F, et al. Regulation of Dual-Specificity Phosphatase (DUSP) Ubiquitination and Protein Stability. Int J Mol Sci. 2019; 20(11): pii: E2668.
2. Shin S-H, et al. Down-Regulation of Dual-Specificity Phosphatase 5 in Gastric Cancer by Promoter CpG Island Hypermethylation and Its Potential Role in Carcinogenesis. Am J Pathol. 2013; 182(4):1275-1285.
3. Virani S, et al. Cancer Epigenetics: A Brief Review. ILAR J. 2012; 53(3-4):359-369.

### SUMMARY OF THE INVENTION

### Problems to be Solved by the Invention

It is an object of the present invention to identify a biomarker that predicts a blood cancer patient to whom the effect of monotherapy using a drug comprising a DNA methyl transferase inhibitor and combination therapy using the drug together with another epigenetic controller can be expected and provide a method for identifying or selecting a blood cancer patient who may benefit from these treatments and a method for predicting the therapeutic effect in the blood cancer patient. It is yet another object of the present invention to provide a pharmaceutical composition for use in the treatment of a patient identified by the method of the present invention and a kit for identifying the patient.

### Means for Solving the Problems

The present inventors have found that the expression amount of DUSP5 in a blood cancer patient decreases as compared with that in a healthy subject, and in cases where a monotherapy using a drug comprising a DNA methyl transferase inhibitor and a combination therapy using the drug together with another epigenetic controller, which exhibit excellent therapeutic effects on blood cancer, are performed in blood cancer patients, the expression amount of DUSP5 increases. That is, the present inventors have found that the expression amount of DUSP5 in blood cancer patients can be used as a biomarker for predicting the drug effect, and completed the present invention.

That is, the objects of the present invention are achieved by the following invention.
[1] A method for identifying or selecting a blood cancer patient who may benefit from the treatment using a drug comprising one or more DNA methyl transferase inhibitors, comprising a step of measuring the expression amount of DUSP5 in a sample obtained from the blood cancer patient, wherein the patient is identified or selected as a blood cancer patient who may benefit from the treatment using the drug when the expression amount of DUSP5 measured in the step is low compared to reference expression amount of DUSP5.
[2] A method for predicting the therapeutic effect of a drug comprising one or more DNA methyl transferase inhibitors in a blood cancer patient, comprising a step of measuring the expression amount of DUSP5 in a sample obtained from the blood cancer patient, wherein the patient is indicated to benefit highly possibly from the treatment using the drug when the expression amount of DUSP5 measured in the step is low compared to reference expression amount of DUSP5.
[3] A method for predicting the therapeutic effect of a drug comprising one or more DNA methyl transferase inhibitors in a blood cancer patient, comprising a step of measuring the expression amount of DUSP5 in a sample obtained from the blood cancer patient, wherein the patient is indicated to benefit highly possibly from the treatment using the drug when the expression amount, which is low compared to reference expression amount of DUSP5 before the treatment using the drug, is increased due to the treatment using the drug.
[4] A method for predicting the therapeutic effect of a drug comprising one or more DNA methyl transferase inhibitors in a blood cancer patient, comprising a step of measuring the expression amount of DUSP5 in a sample obtained from the blood cancer patient, wherein the patient is indicated to be benefiting highly possibly from the treatment using the drug when the expression amount, which is low compared to reference expression amount of DUSP5 before the treatment using the drug, is increased due to the treatment using the drug.
[5] The method according to [1] or [2], wherein the expression amount of DUSP5 measured in the step is decreased by at least about 10% compared to the reference expression amount.
[6] The method according to [1] or [2], wherein the expression amount of DUSP5 measured in the step is decreased by at least about 25% compared to the reference expression amount.
[7] The method according to [1] or [2], wherein the expression amount of DUSP5 measured in the step is decreased by at least about 50% compared to the reference expression amount.
[8] The method according to [1] or [2], wherein the expression amount of DUSP5 measured in the step is decreased by at least about 75% compared to the reference expression amount.
[9] The method according to [1] or [2], wherein the expression amount of DUSP5 measured in the step is decreased by at least about 90% compared to the reference expression amount.
[10] The method according to [3] or [4], wherein with respect to the expression amount of DUSP5 measured in the step, the expression amount, which was low compared to the reference expression amount of DUSP5 before the treatment using the drug, is increased to the same level as the reference expression amount of DUSP5 due to the treatment using the drug.
[11] The method according to [3] or [4], wherein with respect to the expression amount of DUSP5 measured in the step, the expression amount, which was low compared to the reference expression amount of DUSP5 before the treatment using the drug, is increased to a range of about ±20% of the reference expression amount of DUSP5 due to the treatment using the drug.
[12] The method according to any one of [1] to [11], wherein the DNA methyl transferase inhibitor is a compound represented by formula (I):

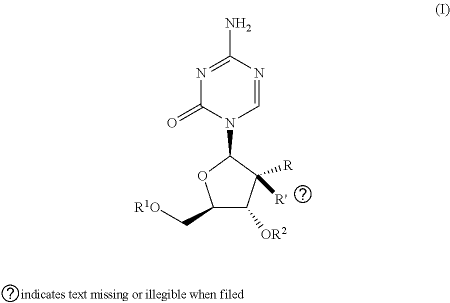

(wherein, R and R′ are each an OR³ group, a hydrogen atom, a halogen atom, or an alkyl group, and R¹, R² and R³ are each a hydrogen atom or a silyl group represented by formula (II):

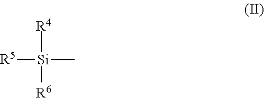

(wherein, R⁴, R⁵ and R⁶ are each an alkyl group, an aryl group, or an arylalkyl group, each of the group may have a substituent.)) or a pharmaceutically acceptable salt thereof, decitabine, a pharmaceutic adjuvant of decitabine with its metabolic enzyme inhibitor, azacytidine, RG-108, thioguanine, zebularine, SGI-110, SGI-1027, lomeguatrib, or procainamide hydrochloride.
[13] The method according to [12], wherein the compound represented by formula (I) is a compound (referred to as OR21 hereinafter) in which R, R¹ and R² are each a hydrogen atom, and R¹ is a triethylsilyl group.
[14] The method according to [12], wherein the pharmaceutic adjuvant of decitabine with its metabolic enzyme inhibitor is ASTX727.
[15] The method according to any one of [1] to [14], wherein the drug further comprises one or more histone methyl transferase inhibitors.
[16] The method according to [15], wherein the histone methyl transferase inhibitor is EPZ-6438, DS-3201b, GSK-126, Chaetocin, or BIX-01294.
[17] The method according to [15], wherein the histone methyl transferase inhibitor is EPZ-6438 and/or DS-3201b.
[18] The method according to any one of [1] to [17], wherein the blood cancer is adult T-cell leukemia/lymphoma, acute myeloid leukemia, acute lymphocytic leukemia, chronic myeloid leukemia, chronic lymphocytic leukemia, or myelodysplastic syndrome.
[19] The method according to any one of [1] to [17], wherein the blood cancer is adult T-cell leukemia/lymphoma or chronic myeloid leukemia.
[20] The method according to any one of [1] to [17], wherein the blood cancer is adult T-cell leukemia/lymphoma.
[21] The method according to any one of [1] to [20], wherein the sample obtained from the patient is a whole blood of the blood cancer patient.
[22] The method according to any one of [1] to [20], wherein the sample obtained from the patient is mononuclear cells in peripheral blood of the blood cancer patient.
[23] The method according to any one of [1] to [22], wherein the benefit of the treatment using the drug is an extension of progression-free survival (PFS).
[24] The method according to any one of [1] to [22], wherein the benefit of the treatment using the drug is an extension of overall survival (OS).
[25] The method according to any one of [1] to [22], wherein the benefit of the treatment using the drug is an increase in overall response rate (ORR).
[26] The method according to any one of [1] to [25], wherein the expression amount of DUSP5 is an expression amount of DUSP5 mRNA.
[27] The method according to [26], wherein the expression amount of mRNA is measured by using at least one method selected from the group consisting of real-time PCR, gene expression profiling, and microarray analysis.
[28] The method according to any one of [1] to [25], wherein the expression amount of DUSP5 is an expression amount of DUSP5 protein.
[29] The method according to [28], wherein the expression amount of protein is measured by using at least one method selected from the group consisting of immunohistochemistry, immunofluorescence, mass spectrometry, flow cytometry, and Western Blotting.
[30] A pharmaceutical composition comprising one or more DNA methyl transferase inhibitors for use in the treatment of a blood cancer patient identified or selected based on the expression amount of DUSP5.
[31] A pharmaceutical composition comprising one or more DNA methyl transferase inhibitors for use in the treatment of a blood cancer patient identified or selected by the method according to [1].
[32] A pharmaceutical composition comprising one or more DNA methyl transferase inhibitors for use in a blood cancer patient to whom the therapeutic effect of a drug is predicted by the method according to any one of [2] to [4].
[33] The pharmaceutical composition according to any one of [30] to [32], wherein the DNA methyl transferase inhibitor is OR21, decitabine, azacytidine, RG-108, thioguanine, zebularine, SGI-110, SGI-1027, lomeguatrib, or procainamide hydrochloride.
[34] The pharmaceutical composition according to any one of [30] to [32], wherein the DNA methyl transferase inhibitor is OR21.
[35] The pharmaceutical composition according to any one of [30] to [34], wherein the pharmaceutical composition further comprises one or more histone methyl transferase inhibitors.
[36] The pharmaceutical composition according to [35], wherein the histone methyl transferase inhibitor is EPZ-6438, DS-3201b, GSK-126, Chaetocin, or BIX-01294.
[37] A kit for identifying or selecting a blood cancer patient who may benefit from the treatment using a drug comprising one or more DNA methyl transferase inhibitors or a drug further comprising one or more histone methyl transferase inhibitors using the method according to [1], the kit comprising a reagent for measuring the expression amount of DUSP5 mRNA in a sample obtained from the patient.
[38] A kit for identifying or selecting a blood cancer patient who may benefit from the treatment using a drug comprising one or more DNA methyl transferase inhibitors or a drug further comprising one or more histone methyl transferase inhibitors using the method according to [1], the kit comprising a reagent for measuring the expression amount of DUSP5 protein in a sample obtained from the patient.
[39] The kit for predicting the therapeutic effect in a blood cancer patient of a drug comprising one or more DNA methyl transferase inhibitors or a drug further comprising one or more histone methyl transferase inhibitors using the method according to any one of [2] to [4], the kit comprising a reagent for measuring the expression amount of DUSP5 mRNA in a sample obtained from the patient.
[40] The kit for predicting the therapeutic effect in a blood cancer patient of a drug comprising one or more DNA methyl transferase inhibitors or a drug further comprising one or more histone methyl transferase inhibitors using the method according to any one of [2] to [4], the kit comprising a reagent for measuring the expression amount of DUSP5 protein in a sample obtained from the patient.

### Effects of the Invention

According to the method of the present invention which uses DUSP5 as a biomarker, it is possible to identify or select a blood cancer patient who may benefit from the treatment using a drug comprising a DNA methyl transferase inhibitor. In addition, according to the method of the present invention, it is possible to predict the therapeutic effect in a blood cancer patient of the drug comprising a DNA methyl transferase inhibitor. Therefore, the indicator that it is highly likely that the therapeutic effect of the drug is exhibited to the blood cancer patient, and the indicator that the therapeutic effect of the drug is exhibiting in the blood cancer patient are provided. Furthermore, by using the pharmaceutical composition of the present invention for use in the treatment of blood cancer patients and the kit of the present invention for identifying the blood cancer patients to be treated, tailor-made medicine for blood cancer patients becomes possible.

### BRIEF DESCRIPTION OF THE DRAWINGS

FIG. 1 shows the results of analyzing the expression amounts of DUSP5 mRNA of patients suffering from adult T-cell leukemia/lymphoma (ATL) and healthy subjects. The mononuclear cells in peripheral blood of ATL patients (smoldering ATL, chronic ATL, and acute ATL) and CD4⁺ T cells in peripheral blood of healthy subjects were used as samples for the measurement. Data analysis was carried out with boxplot. Significant difference between groups was detected by T tests, which were carried out when the variance was equal in the group of healthy subjects vs the group of patients with smoldering ATL and the group of healthy subjects vs the group of patients with chronic ATL, and when the variance was different in the group of healthy subjects vs the group of patients with acute ATL.

FIG. 2A shows the results of measuring the effects of DNA methyl transferase inhibitor and histone methyl transferase inhibitor on the expression amount of DUSP5 gene in MT-1 cells (ATL-derived cell lines). OR21 was used as a DNA methyl transferase inhibitor, and EPZ-6438 (shown as EPZ in the figure) was used as a histone methyl transferase inhibitor. The control was the group without the addition of OR21 and EPZ-6438.

FIG. 2B shows the results of measuring the effects of DNA methyl transferase inhibitor and histone methyl transferase inhibitor on the expression amount of DUSP5 protein in MT-1 cells (ATL-derived cell lines) by Western blotting. 0.5 μM OR21 was used as a DNA methyl transferase inhibitor, and 2.5 μM EPZ-6438 (shown as EPZ in the figure) was used as a histone methyl transferase inhibitor. β-actin was used as a loading control. Ctrl represents a group in which none of the above inhibitors were added, and Comb represents a group in which the both inhibitors were added at the concentration described above.

FIG. 3 shows the results of measuring the influence of DNA methyl transferase inhibitor and histone methyl transferase inhibitor on proliferation of MT-1 cells (ATL-derived cell lines). OR21 was used as a DNA methyl transferase inhibitor, and EPZ-6438 (shown as EPZ in the figure) and DS-3201b (shown as DS in the figure) were used as histone methyl transferase inhibitors.

### EMBODIMENTS FOR CARRYING OUT THE INVENTION

### [DNA Methyl Transferase Inhibitors]

The DNA methyl transferase is a group of enzymes which methylate the N6-position of adenine, the N4-position of cytosine, or the 5-position of cytosine in DNA chains. Particularly, in the part of sequence called CpG islands and often recognized in a promoter region of an expression gene, the group of enzyme which methylate 5-position of cytosine plays an extremely important role in regulating normal generation and differentiation of cells. Since the DNA methyl transferase has epigenetic effect on gene expression, the inhibitor against the enzyme is used as an anticancer agent.

Examples of the DNA methyl transferase inhibitor used in the present invention include a compound represented by formula (I) or a pharmaceutically acceptable salt thereof, decitabine, azacytidine, RG-108, thioguanine, zebularine, SGI-110, SGI-1027, lomeguatrib, and procainamide hydrochloride, but are not limited thereto.

Examples of preferable combinations of R and R′ in formula (I) are as follows: (i) the combination in which R is an OR³ group, a hydrogen atom, a halogen atom, or an alkyl group, and R′ is a hydrogen atom, (ii) the combination in which R is an OR³ group or a hydrogen atom, and R′ is a hydrogen atom, and (iii) the combination in which R is a fluorine atom, and R′ is a fluorine atom.

An “alkyl group” refers to, unless otherwise limited, a saturated aliphatic hydrocarbon group such as a linear or branched or cyclic alkyl group having 1 to 8 carbon atoms. Examples thereof include C₁-C₆ alkyl group such as methyl, ethyl, propyl, isopropyl, butyl, sec-butyl, isobutyl, tert-butyl, pentyl, hexyl groups, and the like, heptyl, 2-methylhexyl, 5-methylhexyl, 2,2-dimethylpentyl, 4,4-dimethylpentyl, 2-ethylpentyl, 1,1,3-trimethylbutyl, 1,2,2-trimethylbutyl, 1,3,3-trimethylbutyl, 2,2,3-trimethylbutyl, 2,3,3-trimethylbutyl, 1-propylbutyl, 1,1,2,2-tetramethylpropyl, octyl, 2-methylheptyl, 3-methylheptyl, 6-methylheptyl, 2-ethylhexyl, 5,5-dimethylhexyl, 2,4,4-trimethylpentyl, 1-ethyl-1-methylpentyl, cyclopropyl, cyclobutyl, cyclopentyl, cyclohexyl, cycloheptyl, cyclooctyl groups, and the like, preferably, a C₁-C₆ alkyl group. Preferable examples of the C₁-C₆ alkyl group include methyl, ethyl, and propyl groups. In addition, preferable examples of the cyclic alkyl group include cyclopentyl and cyclohexyl groups.

An “aryl group” refers to a monocyclic or bicyclic aromatic hydrocarbon, preferably a C₆-C₁₀ aryl group such as phenyl or naphthyl group, and more preferably phenyl group.

An “arylalkyl group” refers to an alkyl group substituted by an aryl, preferably a C₇-C₁₄ arylalkyl group. Examples of the C₇-C₁₄ arylalkyl group include, but are not limited to, benzyl, phenethyl, naphthylmethyl groups, and the like.

“An alkyl group which may have a substituent, an aryl group which may have a substituent, or an arylalkyl group which may have a substituent” refer to those which may have a substituent or may be unsubstituted. In the case of being substituted, there may be 1 to 5 substituents, preferably 1 to 3 substituents at possible positions of the alkyl, aryl or arylalkyl groups. When the number of substituents is 2 or more, each substituent may be the same or different. Examples of the substituent include halogen atoms, alkyl, cyano, and nitro groups, etc., preferably alkyl groups or halogen atoms.

A “halogen atom” refers to fluorine, chlorine, bromine, or iodine atoms, and the like, preferable examples include fluorine and chlorine atoms.

The compound represented by formula (I) is preferably a compound (referred to as OR21) in which R, R¹ and R² are each a hydrogen atom and R¹ is a triethylsilyl group. OR21 has the chemical name of 5′-O-triethylsilyl-2′-deoxy-5-azacytidine, and has the structure represented by formula (III). The production of a compound represented by formula (I) can be carried out in accordance with the method shown by WO2017/183217.

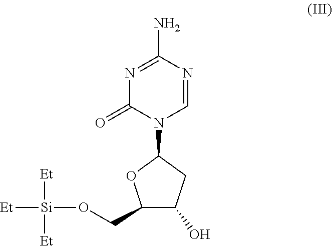 [SMILES: CC[Si](CC)(CC)OC[C@H]1O[C@@H](n2cnc(N)nc2=O)C[C@@H]1O]

Examples of the pharmaceutically acceptable salts of compounds represented by formula (I) include alkali metal salts (for example, sodium salts and potassium salts), alkaline earth metal salts (for example, magnesium salts and calcium salts), ammonium salts, mono-, di-, or tri-, lower (alkyl or hydroxyalkyl) ammonium salts (for example, ethanol ammonium salts, diethanol ammonium salts, triethanol ammonium salts, trometammine salts), hydrochloride, hydrobromate, hydroiodide, nitrate, phosphate, sulfate, formate, acetate, citrate, oxalate, fumarate, maleate, succinate, malate, tartrate, trichloroacetate, trifluoroacetate, methanesulfonate, benzenesulfonate, p-toluenesulfonate (tosilate), mesitylene sulfonate, and naphthalenesulfonate. In addition, the salt may be an anhydride or a solvate. Examples of the solvate include hydrate, solvates of methanol, ethanol, propanol, and 2-propanol.

Decitabine has the chemical name of 4-amino-1-(2-deoxy-p-D-erythro-pentofuranosyl)-1,3,5-triazin-2(1H)-one and CAS number of 2353-33-5. Examples of the pharmaceutic adjuvant of decitabine and its metabolic enzyme inhibitor include ASTX727. ASTX727 is a pharmaceutic adjuvant of decitabine and E7727 (generic name: cedazuridine), which is a cytidine deaminase inhibitor. E7727 has the chemical name of (4R)-1-(2-deoxy-2,2-difluoro-beta-D-erythro-pentofuranosyl)-4-hydroxytetrahydropyrimidin-2(1H)-one and CAS number of 1141397-80-9. Azacytidine has the chemical name of 4-amino-1-β-D-ribofuranosyl-s-triazin-2(1H)-one and CAS number of 320-67-2. RG-108 has the chemical names of 2(S)-(1,3-dioxo-2,3-dihydro-1H-isoindol-2-yl)-3-(1H-indol-3-yl)propionic acid and CAS number of 48208-26-0. Tioguanine has the chemical name of 2-amino-1,9-dihydro-6H-purine-6-thione and CAS number of 154-42-7. Zebularine has the chemical name of 1-(β-D-ribofuranosyl)pyrimidin-2(1H)-one and CAS number of 3690-10-6. SGI-110 (generic name: guadecitabine) has the chemical name of 2′-deoxy-5′-O-[(2′-deoxy-5-azacytidin-3′-O-yl)(hydroxy)phosphoryl]guanosine and CAS number of 929904-85-8 (sodium salt). SGI-1027 has the chemical name of N-[4-(2-amino-6-methylpyrimidin-4-ylamino)phenyl]-4-(quinolin-4-ylamino)benzamide and CAS number of 1020149-73-8. Lomeguatrib has the chemical name of 6-[(4-bromo-2-thienyl)methoxy]-7H-purin-2-amine and CAS number of 192441-08-0. These compounds may be in the form of pharmaceutically acceptable salts thereof. The pharmaceutically acceptable salts are exemplified by the above-mentioned salts. The salts may be the anhydrides or solvates described above.

### [Histone Methyl Transferase Inhibitors]

The histone methyl transferase is an enzyme which transfers a methyl group from S-adenosylmethionine, a coenzyme, to the amino group of lysine residue of histone 3 (H3) protein. The methylation modification of lysine residue has an epigenetic effect on gene expression, and is thus extremely important for gene expression regulation. Therefore, the inhibitor against histone methyl transferase is used as an anticancer agent.

Examples of the histone methyl transferase inhibitor used in the present invention include, but are not limited thereto, EPZ-6438, DS-3201b, GSK-126, Chaetocin, and BIX-01294, etc., and preferably EPZ-6438 and DS-3201b.

EPZ-6438 (generic name: tazemetostat) is an inhibitor against histone methyl transferase EZH2. EPZ-6438 has the chemical name of N-[(4,6-dimethyl-2-oxo-1,2-dihydropyridin-3-yl) methyl]-5-[ethyl(tetrahydro-2H-pyran-4-yl) amino]-4-methyl-4′-(morpholin-4-ylmethyl) biphenyl-3-carboxamide and CAS number of 1467052-75-0 (hydrobromide salt). DS-3201b (generic name: valemetostat) is a daul inhibitor against histone methyl transferases EZH1 and EZH2. DS-3201b has the chemical name of 4-methylbenzene-1-sulfonic acid (2R)-7-chloro-2-[(trans)-4-(dimethylamino) cyclohexyl]-N-[(4,6-dimethyl-2-oxo-1,2-dihydropyridin-3-yl) methyl]-2,4-dimethyl-2H-1,3-benzodioxole-5-carboxamide and CAS number of 1809336-39-7 (tosilate). GSK-126 is an inhibitor against histone methyl transferase EZH2. GSK-126 has the chemical name of 1-[2(S)-butyl]-N-(4,6-dimethyl-2-oxo-1,2-dihydropyridin-3-ylmethyl)-3-methyl-6-[6-(1-piperazinyl) pyridin-3-yl]-1H-indole-4-carboxamide and CAS number of 1346574-57-9. Chaetocin has the chemical name of (3S,3'S,5aR,5′aR,10bR,10′bR,11aS,11′aS)-1,1′,2,2′,3,3′,4,4′,5a,5′a,6,6′,10b,10b′,11,11′,11a,1a′-octadecahydro-3,3′-bis(hydroxymethyl)-2,2′-dimethyl-[bi-3,11a-epidithio-11aH-pyrazino[1′,2′:1,5]pyrrolo[2,3-b]indole]-1,1′,4,4′-tetraone and CAS number of 28097-03-2. BIX-01294 has the chemical name of N-(1-benzylpiperidin-4-yl)-6,7-dimethoxy-2-(4-methylperhydro-1,4-diazepin-1-yl) quinazolin-4-amine and CAS number of 935693-62-2.

### [Measurement of Expression Amount of DUSP5 in Blood Cancer Patients]

In the method of the present invention, a “blood cancer patient” refers to a patient diagnosed with adult T cell leukemia/lymphoma, acute myeloid leukemia, acute lymphocytic leukemia, chronic myeloid leukemia, chronic lymphocytic leukemia, or myelodysplastic syndrome. However, he (she) may also be a patient suffering from other type of cancer. In one embodiment, in the method of the present invention, a blood cancer patient is preferably one who suffers from adult T-cell leukemia/lymphoma or chronic myeloid leukemia, and more preferably one who suffers from adult T-cell leukemia/lymphoma. A “healthy subject” refers to a subject who is not suffering from blood cancer or is not experiencing any signs or symptoms of blood cancer.

The method of the present invention comprises a step of measuring the expression amount of DUSP5 in a sample obtained from a blood cancer patient. A “sample” refers to the tissue which comprises the cells of a blood cancer patient. Examples of collection source of the sample include any tissue in the body, such as whole blood, umbilical cord blood, lymph, tissue fluid (interstitial fluid, intercellular fluid, and interstitial fluid), body cavity fluid (ascitic fluid, pleural fluid, pericardial fluid, articular fluid, synovial fluid, and aqueous humor), nose juice, and the like. However, peripheral blood is preferable, and mononuclear cells in peripheral blood are more preferable, due to less invasiveness to patients. The mononuclear cells in peripheral blood can be obtained from the whole blood collected by Ficoll density gradient centrifugation, for example. In addition, cells which are expressed or not expressed by specific marker protein on cell surface may be separated and collected by positive or negative selection using magnetic beads for cell separation. Cells of a blood cancer patient may be a cell line established from separated or isolated cells from a tissue comprising the cells of the blood cancer patient.

The “expression amount of DUSP5” is the expression amount of DUSP5 mRNA or the expression amount or DUSP5 protein in samples. In order to measure the expression amount of mRNA in samples, total RNA is usually extracted from tissues. Methods for extracting total RNA are well known to a person skilled in the art. As methods of detecting the expression amount of DUSP5 mRNA, any method may be used as long as a part or all of DUSP5 mRNA or single strand complementary DNA (cDNA) can be detected specifically. Following examples can be given: a method of extracting the total RNA of cells present in a sample and detecting by Northern blotting using a probe consisting of base sequences complementary to DUSP5 mRNA, a method of synthesizing cDNA from the extracted total RNA using reverse transcriptase and then detecting by a quantitative PCR method such as a competitive PCR (polymerase chain reaction) method or a real-time PCR method, etc., and a method of synthesizing cDNA from the total RNA using reverse transcriptase, then labeling the cDNA with biotin or digoxigenin, etc., indirectly labeling the cDNA with an antibody that recognizes avidin or digoxigenin, which has a high affinity for biotin labeled with a fluorescent substance, and then detecting with a microarray using a probe consisting of base sequences complementary to cDNA which are immobilized on a support such as glass, silicon, plastic, etc. that can be used for hybridization, and synthesized from the DUSP5 mRNA and the mRNA of any reference gene. By gene expression profiling, the DUSP5 mRNA expression can be profiled, and its relation with signs or symptoms in a blood cancer patient can also be examined in more detail.

In order to measure the expression amount of DUSP5 protein in samples, methods understood by a person skilled in the art, such as immunohistochemistry, immunofluorescence, mass spectrometry, flow cytometry, Western blotting, etc. can be used. As anti-DUSP5 antibodies required for the detection of DUSP5 protein, those which are commercially available can be used. In mass spectrometry, for a sample solution, it is preferable to use an ionization method such as MALDI-MS (matrix assisted laser desorption ionization mass spectrometry) by which compounds of high molecular weight are less likely to decompose.

The expression amount of DUSP5, which is a biomarker, can be normalized by taking the expression amount of internal standard as a comparison control. The measurement of the increase or decrease in the expression amount of DUSP5 may be performed by comparing normalized expression amount of DUSP5 in multiple samples including samples from a blood cancer patient and a healthy subject. The internal standard is a biomarker for measuring the same sample as the sample in which the expression amount of DUSP5 is measured, preferably a housekeeping biomarker. The housekeeping biomarker is the protein that is a housekeeping gene and a translation product thereof, and examples thereof include GAPDH (glyceraldehyde-3-phosphate dehydrogenase) gene, R-actin gene, and translation protein thereof. The housekeeping gene is a gene encoding a protein typically present in all cell types. Its activity is essential for maintaining cell functions.

The “reference expression amount of DUSP5” refers to an expression amount of DUSP5 in a sample collected from a healthy subject which is not a blood cancer patient. A reference expression amount of DUSP5 can be an expression amount of DUSP5 mRNA or an expression amount of DUSP5 protein. A reference expression amount of DUSP5 can be normalized by taking the internal standard as a comparison control. The comparison between an expression amount of DUSP5 in a blood cancer patient and a reference expression amount of DUSP5 in a healthy subject is performed between the biomarkers of the same type (mRNA or protein). It is preferable that a reference expression amount of DUSP5 is measured in multiple healthy subjects before measurement of an expression amount of DUSP5 in a blood cancer patient, and its database is created in advance.

A case where an expression amount of DUSP5 in a sample obtained from a blood cancer patient is “low” compared to a reference expression amount of DUSP5 refers to the case where an expression amount of DUSP5 in a sample from a blood cancer patient is lower than a reference value which is taken as the median or average of the reference expression amount of DUSP5. In some embodiments, a case where an expression amount of DUSP5 in a sample is “low” refers to a case where the expression amount of DUSP5 in a sample decreases by about 10%, about 25%, about 50%, about 75%, or about 90%, as compared with the reference value. Whether the median or the average of a reference expression amount of DUSP5 is taken as the reference value can be measured based on the distribution of the expression amount of DUSP5 in multiple healthy subjects. A person skilled in the art can select a reference value estimated to be more appropriate based on the presence or absence of outliers in the distribution of expression amounts of DUSP5. The term “about” refers to a normal error range of each value that can be easily known by a person skilled in the art.

When an expression amount of DUSP5 in a sample obtained from a blood cancer patient shows a lower value than the reference value, the blood cancer patient may be stratified as a patient who benefits from the treatment using the drug even before the treatment using the drug. In addition, the expression amount of DUSP5 can be used as a biomarker that predicts the possibility that the blood cancer patient may benefit from the treatment using the drug even before the treatment using the drug.

In the method of the present invention, the treatment using the drug comprises single, intermittent, or continuous administration of the drug. By measuring an expression amount of DUSP5 of a blood cancer patient administered with the drug at an arbitrary time point after the administration of the drug, and observing the process of increase, due to the treatment using the drug, in the expression amount of DUSP5 of the blood cancer patient who had a lower value than the reference expression amount of DUSP5, an indicator of the possibility that the patient will benefit from the treatment using the drug can be achieved. It is also possible to measure an appropriate dose of the drug based on the indicator relating to the possibility of benefiting from the treatment.

The expression amount of DUSP5 of a blood cancer patient who had a lower value compared to a reference expression amount of DUSP5 may increase to the same level as the reference expression amount of DUSP5. In one embodiment, the increase to the same level means an increase to the range of about ±20% of the reference expression amount of DUSP5.

The “benefit from a treatment using a drug” and “therapeutic effect of a drug on a blood cancer patient” are medically useful or preferable response occurring in a blood cancer patient due to treatment using a drug, such as progression-free survival (PFS), overall survival (OS), and overall response rate (ORR), which are used as clinical trial endpoints (outcome measures which indicate therapeutic efficacy) in the development of anticancer drugs. PFS refers to a period in which the stage of cancer remains stable without progression during or after treatment. OS refers to a period since the start of a treatment in which a patient has survived. ORR refers to the percentage of patients who have been shown to have a therapeutic effect. Endpoints except PFS, OS, and ORR can also be used.

### [A Pharmaceutical Composition]

The present invention provides a pharmaceutical composition comprising one or more DNA methyl transferase inhibitors or a pharmaceutical composition further comprising one or more histone methyl transferase inhibitors for use in the treatment of a blood cancer patient identified or selected by the method of the present invention and for use in a blood cancer patient in which the therapeutic effect of the drug is predicted by the method of the present invention. The pharmaceutical composition may comprise a pharmaceutically acceptable carrier.

The pharmaceutical composition of the present invention may be in a dosage form for oral or parenteral use. Examples of the dosage form for oral use include solid preparations such as tablets, capsules, granules, powder, and the like. Examples of the dosage form for parenteral use include an injection, an inhalant, a suppository, a patch, and the like.

Examples of the pharmaceutically acceptable carrier include a solvent such as sterilized water or physiological saline, etc., a binder such as gelatin, corn starch, tragant gum, gum Arabic, etc., an excipient such as crystalline cellulose, etc., and a swelling agent such as alginic acid, etc.

The pharmaceutical composition of the present invention may further comprise an additive. Examples of the additive include a lubricant such as magnesium stearate, etc., a sweetener such as sucrose, lactose, saccharin, etc., a flavor agent such as peppermint, Gaultheria adenothrix oil, etc., a stabilizer such as benzyl alcohol, phenol, a buffer agent such as phosphate, sodium acetate, etc., a dissolution aid such as benzyl benzoate, benzyl alcohol, etc., an antioxidant, a preservative, etc.

The pharmaceutical composition of the invention can be formulated by appropriately mixing with the carrier and additive described above in a form of generally accepted unit dose required for pharmaceutical practices.

The pharmaceutical composition of the present invention can be administered to a patient as an intrathecal injection, intraarterial injection, intravenous injection, subcutaneous injection, etc., and additionally by intranasal, transbronchial, intramuscular, percutaneous or oral methods known to a person skilled in the art. The dosage varies depending on the body weight, age, signs or symptoms of a patient, the method of administration, and the like. However, an appropriate dosage can be appropriately selected by a person skilled in the art.

When the pharmaceutical composition of the present invention is administered to a patient, a pharmaceutical composition comprising multiple DNA methyl transferase inhibitors may be administered as a single formulation, or pharmaceutical compositions each comprising a DNA methyl transferase inhibitor may be administered as multiple formulations. When multiple formulations are administered, they may be administered at the same time or at a time interval.

In one embodiment, when the pharmaceutical composition of the present invention further comprises one or more histone methyl transferase inhibitors, the case where it is (they are) comprised with DNA methyl transferase inhibitor in the same formulation, and the case where it is (they are) comprised with DNA methyl transferase inhibitor in different formulations are included.

In one embodiment, the present invention provides a method for treating blood cancer, comprising administering a pharmaceutical composition comprising an effective amount of one or more DNA methyl transferase inhibitors to a blood cancer patient who may benefit from the treatment identified by the method of the present invention. In one embodiment, the pharmaceutical composition may further comprise one or more histone methyl transferase inhibitors.

In one embodiment, the present invention provides a pharmaceutical composition comprising a DNA methyl transferase inhibitor for treating the blood cancer in a blood cancer patient who may benefit from the treatment identified by the method of the present invention. In one embodiment, the pharmaceutical composition may further comprise one or more histone methyl transferase inhibitors.

In one embodiment, the present invention provides a pharmaceutical composition comprising a DNA methyl transferase inhibitor for producing a therapeutic agent for blood cancer in a blood cancer patient who may benefit from the treatment identified by the method of the present invention. In one embodiment, the pharmaceutical composition may further comprise one or more histone methyl transferase inhibitors.

The kit for identifying or selecting a blood cancer patient who may benefit from the treatment using the drug comprising one or more DNA methyl transferase inhibitors or the drug further comprising one or more histone methyl transferase inhibitors using the method of the present invention, or the kit for predicting a therapeutic effect in a blood cancer patient of the drug comprising one or more DNA methyl transferase inhibitors or the drug further comprising one or more histone methyl transferase inhibitors using the method of the present invention comprises reagents for measurement of expression amounts of DUSP5 and mRNA of an internal standard, when necessary, and an instruction for identifying a blood cancer patient who may benefit from the treatment or an instruction for predicting a therapeutic effect in the blood cancer patient. The composition of the reagents can be appropriately measured by a person skilled in the art based on the measurement method of mRNA. For example, the kit comprises a reagent for extracting total RNA from a sample obtained from a blood cancer patient, a reagent for synthesizing cDNA from the total RNA, a reagent for carrying out PCR, and a reagent for detecting a PCR amplification product.

The kit for identifying or selecting a blood cancer patient who may benefit from the treatment using the drug comprising one or more DNA methyl transferase inhibitors or the drug further comprising one or more histone methyl transferase inhibitors using the method of the present invention, or the kit for predicting a therapeutic effect in a blood cancer patient of the drug comprising one or more DNA methyl transferase inhibitors or the drug further comprising one or more histone methyl transferase inhibitors using the method of the present invention comprises reagents for measurement of expression amounts of DUSP5 and protein of an internal standard, when necessary, and an instruction for identifying a blood cancer patient who may benefit from the treatment or an instruction for predicting a therapeutic effect in the blood cancer patient. The composition of the reagents can be appropriately measured by a person skilled in the art based on the measurement method of protein. For example, the kit comprises a reagent for extracting protein from a sample obtained from a blood cancer patient and a reagent for detecting a target protein by ELISA (enzyme-linked immuno sorbent assay).

Hereafter, the present invention will be described with reference to examples. However, the present invention is not limited to the following examples.

### Example 1

[Expression Amounts of DUSP5 mRNA of Patients with Adult T-Cell Leukemia/Lymphoma (ATL) and Healthy Subjects]

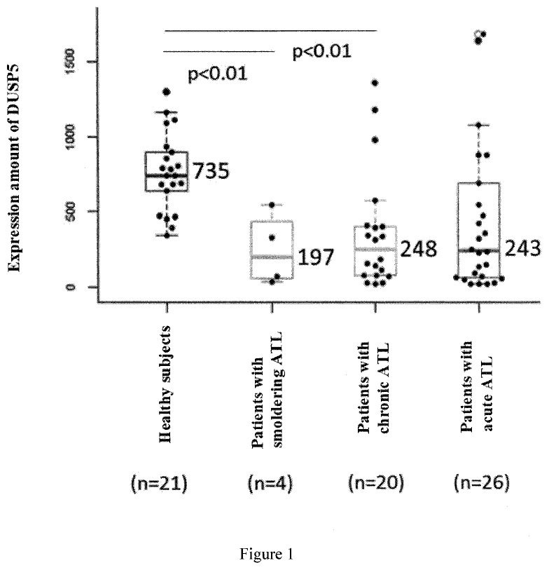

Total RNA was extracted from mononuclear cells in peripheral blood of 4 patients with smoldering ATL, 20 patients with chronic ATL, and 26 patients with acute ATL and CD4⁺ T cells in peripheral blood of 21 healthy subjects, and the results of a comprehensive analysis of expression amounts of mRNA in these cells using the Whole Human Genome DNA Microarray 4×44K (Agilent Technology) have been reported (Yamagishi M, et al. Polycomb-mediated Loss of miR-31 Activates NIK-dependent NF-κB Pathway in Adult T Cell Leukemia and Other Cancers. Cancer Cell. 2012; 21(1):121-135). The present inventors analyzed the results reported by Yamagishi et al. and calculated the expression amount of DUSP5 mRNA in the above cells. The data analysis was carried out with boxplot, and the results were as shown in FIG. 1. It can be seen apparently from FIG. 1 that patients with all types of ATL exhibited significantly lower values of expression amount of mRNA with respect to those of healthy subjects (reference expression amount). In the boxplot, the values on the center line and its right side indicate medians. The uppermost end of the box represents lower quartile, and the lowermost end of the box represents upper quartile. The upper whisker and the lower whisker refer to the maximum data point and the minimum data point, respectively, in the range of [(upper quartile−1.5×(lower quartile−upper quartile)] or more and [(lower quartile]+1.5×(lower quartile)−upper quartile)] or less. The points outside the range are outliers.

It has been shown that the expression amounts of DUSP5 mRNA of patients with smoldering ATL, chronic ATL, and acute ATL are significantly low, compared with the expression amount of DUSP5 mRNA in healthy subjects, that is, the reference expression amount of DUSP5. The medians of the expression amount of DUSP5 mRNA in the patients with ATL were decreased to 197 by 73.2% in the patients with smoldering ATL, to 248 by 66.3% in the patients with chronic ATL, and to 243 by 66.9% in the patients with acute ATL, compared with the median of 735 as the reference expression amount of DUSP5.

### Example 2

[Effects of DNA Methyl Transferase Inhibitor and Histone Methyl Transferase Inhibitor on the Expression Amount of DUSP5 mRNA in Mt-1 Cells]

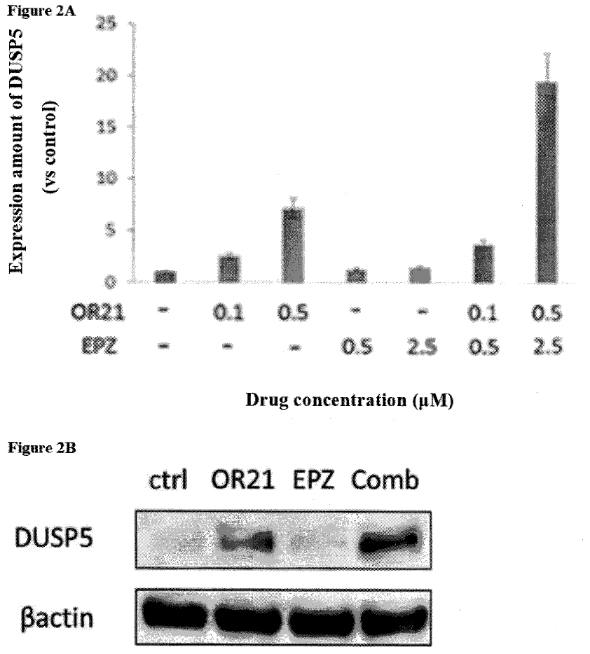

MT-1 cells (ATL-derived cell lines) were purchased from JCRB Cell Bank and cultured in an RPMI1640 culture solution comprising 10% fetal bovine serum (FBS) and 1% penicillin-streptomycin in an environment of 37° C. and 5% CO₂. The culture was started by additions of OR21, a DNA methyl transferase inhibitor and/or EPZ-6438, a histone methyl transferase inhibitor to a culture solution of MT-1 cells, exchanged with a fresh culture solution comprising the inhibitors after 2 days, and further cultured for 2 days. After culturing, the cells were collected and total RNA was extracted, and the expression amount of DUSP5 mRNA was measured by RT-PCR using SYBR @ Green (Takara Bio). The results are shown in FIG. 2A. It was shown that OR21 enhanced DUSP5 mRNA expression in a concentration-dependent manner at 0.1 μM and 0.5 μM. EPZ-6438 (EPZ) had almost no effect on DUSP5 mRNA expression at 0.5 μM and 2.5 μM. On the other hand, it was shown that DUSP5 mRNA expression was synergistically enhanced if OR21 and EPZ-6438 (EPZ) coexisted.

### Example 3

### [Effects of DNA Methyl Transferase Inhibitor and Histone Methyl Transferase Inhibitor on the Expression Amount of DUSP5 Protein in Mt-1 Cells]

MT-1 cells (ATL-derived cell lines) were purchased from JCRB Cell Bank and cultured in an RPMI1640 culture solution comprising 10% fetal bovine serum (FBS) and 1% penicillin-streptomycin in an environment of 37° C. and 5% CO₂. The culture was started by additions of OR21, a DNA methyl transferase inhibitor and/or EPZ-6438, a histone methyl transferase inhibitor to a culture solution of MT-1 cells, exchanged with a fresh culture solution comprising the inhibitors after 2 days, and further cultured for 2 days. After culturing, the cells were collected and dissolved, and the expression amount of DUSP5 protein was measured by Western blotting. The results are shown in FIG. 2B. In case of addition of EPZ-6438 (2.5 μM), a histone methyl transferase inhibitor, almost no effect on the expression amount of DUSP5 protein was confirmed. However, it was shown that when OR21 (0.5 μM), a DNA methyl transferase inhibitor, was added, apparent increase in DUSP5 protein expression was confirmed, and if EPZ-6438 coexisted, the DUSP5 protein expression was synergistically enhanced.

### Example 4

### [Effects of DNA Methyl Transferase Inhibitor and Histone Methyl Transferase Inhibitor on Proliferation of Mt-1 Cells]

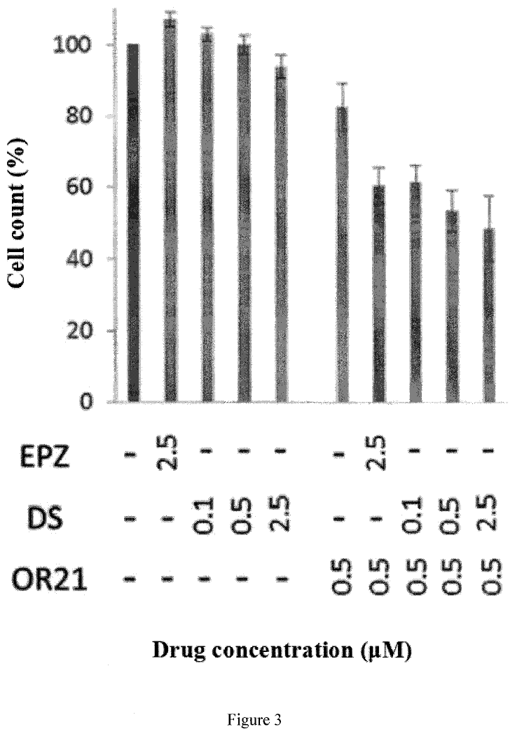

MT-1 cells (ATL-derived cell lines) were purchased from JCRB Cell Bank. MT-1 cells were seeded on a 24-well plate at 0.4×10⁵ cells/0.9 mL/well and cultured for 3 hours in an RPMI1640 culture solution comprising 10% fetal bovine serum (FBS) and 1% penicillin-streptomycin in an environment of 37° C. and 5% CO₂. Then, OR21, a DNA methyl transferase inhibitor and/or EPZ-6438 or DS-3201b, histone methyl transferase inhibitors were dissolved in the same culture solution, and 0.1 mL thereof was added to each well, and cultured for 4 days while exchanging with a fresh culture solution comprising each inhibitor every two days. The final concentrations of the inhibitors in the culture solution were 0.5 μM for OR21, 2.5 μM for EPZ-6438, and 0.1 μM, 0.5 μM, and 2.5 μM for DS-3201b. In addition, groups of single and combined additions of each inhibitor were provided. The effect of each inhibitor on cell proliferation was measured using Cell Countdown Kit-8 (Dojindo Laboratories). The water-soluble formazan in each well produced from reduction of tetrazolium salt WST-1 was measured at two wavelength (450 nm/620 nm) using a plate reader (Varioskan Flash, Thermo-Fisher Scientific). The results are shown in FIG. 3. The results are expressed as relative values with the absorbance of the wells in which no inhibitor was added as 100%.

In the range examined, EPZ-6438 (2.5 μM), a histone methyl transferase inhibitor and DS-3201b (0.1 μM to 2.5 μM), a DNA methyl transferase inhibitor hardly affected the proliferation of MT-1 cells. On the other hand, OR21, a DNA methyl transferase inhibitor exhibited an effect of inhibiting proliferation at 0.5 μM, and when EPZ-6438 or DS-3201b coexisted, the inhibition effect was enhanced. The effect of inhibiting proliferation observed correlated with the effect of enhancing the expression amount of DUSP5 mRNA (FIG. 2A) and the expression amount of protein (FIG. 2B) in MT-1 cells. The result suggested that when DUSP5 expression in a blood cancer patient is low compared with the reference expression amount of healthy subjects, the effect of inhibiting proliferation of cancer cells and the enhancement of DUSP5 expression occur in parallel due to monotherapy using a DNA methyl transferase inhibitor or combination therapy with a histone methyl transferase inhibitor. Since the effect of inhibiting proliferation of cancer cells of drugs associated with the therapeutic effects of drugs on blood cancer patients, identifying and selecting a blood cancer patient whose DUSP5 expression is lower than the reference expression amount of healthy subjects mean identifying or selecting the patient as a blood cancer patient who may benefit from the treatment using the drug.

In a blood cancer patient having DUSP5 expression lower than the reference expression amount of healthy subjects, the inhibition of proliferation of cancer cells may occur in parallel with the enhancement of DUSP5 expression due to a monotherapy using a DNA methyl transferase inhibitor or a combination therapy using a histone methyl transferase inhibitor. Therefore, it is suggested that such blood cancer patients may benefit from the treatment.

It is suggested that in the case where the expression amount of DUSP5 in a blood cancer patient is low compared with the reference expression amount of healthy subjects, and the expression amount of DUSP5 of the blood cancer patient is increased due to a monotherapy using a DNA methyl transferase inhibitor or a combination therapy with a histone methyl transferase inhibitor, the blood cancer patient may highly possibly benefit or is benefiting from the treatment using the drug, based on the correlation between the effect of inhibiting the DUSP5 expression and the effect of inhibiting the proliferation of cancer cells of the drug.

1. A method for identifying or selecting a blood cancer patient who may benefit from the treatment using a drug comprising one or more DNA methyl transferase inhibitors, comprising a step of measuring the expression amount of DUSP5 in a sample obtained from the blood cancer patient, wherein the patient is identified or selected as a blood cancer patient who may benefit from the treatment using the drug when the expression amount of DUSP5 measured in the step is low compared to reference expression amount of DUSP5.

2. A method for predicting the therapeutic effect of a drug comprising one or more DNA methyl transferase inhibitors in a blood cancer patient, comprising a step of measuring the expression amount of DUSP5 in a sample obtained from the blood cancer patient, wherein the patient is indicated to benefit highly possibly from the treatment using the drug when the expression amount of DUSP5 measured in the step is low compared to reference expression amount of DUSP5.

3-4. (canceled)

5. The method according to claim 1, wherein the expression amount of DUSP5 measured in the step is decreased by at least about 10% compared to the reference expression amount.

6. The method according to claim 2, wherein the expression amount of DUSP5 measured in the step is decreased by at least about 10% compared to the reference expression amount.

7-11. (canceled)

12. The method according to claim 1, wherein the DNA methyl transferase inhibitor is a compound represented by formula (I): 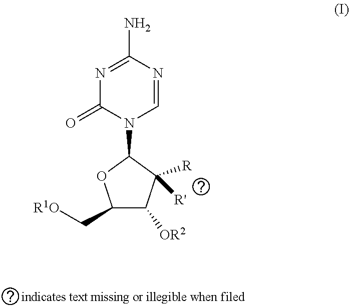
wherein, R and R′ are each an OR³ group, a hydrogen atom, a halogen atom, or an alkyl group, and R¹, R² and R³ are each a hydrogen atom or a silyl group represented by formula (II): 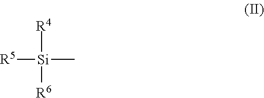
wherein, R⁴, R⁵ and R⁶ are each an alkyl group, an aryl group, or an arylalkyl group, each of the group may have a substituent or a pharmaceutically acceptable salt thereof, decitabine, a pharmaceutic adjuvant of decitabine with its metabolic enzyme inhibitor, azacytidine, RG-108, thioguanine, zebularine, SGI-110, SGI-1027, lomeguatrib, or procainamide hydrochloride.

13. The method according to claim 12, wherein the compound represented by formula (I) is a compound (referred to as OR21 hereinafter) in which R, R¹ and R² are each a hydrogen atom, and R¹ is a triethylsilyl group.

14. The method according to claim 12, wherein the pharmaceutic adjuvant of decitabine with its metabolic enzyme inhibitor is ASTX727.

15. The method according to claim 1, wherein the drug further comprises one or more histone methyl transferase inhibitors.

16. The method according to claim 15, wherein the histone methyl transferase inhibitor is EPZ-6438, DS-3201 b, GSK-126, Chaetocin, or BIX-01294.

17. (canceled)

18. The method according to claim 1, wherein the blood cancer is adult T-cell leukemia/lymphoma, acute myeloid leukemia, acute lymphocytic leukemia, chronic myeloid leukemia, chronic lymphocytic leukemia, or myelodysplastic syndrome.

19-20. (canceled)

21. The method according to claim 1, wherein the sample obtained from the patient is whole blood or mononuclear cells of the blood cancer patient.

22. (canceled)

23. The method according to claim 1, wherein the benefit of the treatment using the drug is an extension of progression-free survival (PFS), an extension of overall survival (OS), or an increase in overall response rate (ORR).

24-25. (canceled)

26. The method according to claim 1, wherein the expression amount of DUSP5 is an expression amount of DUSP5 mRNA or is an expression amount of DUSP5 protein.

27. The method according to claim 26, wherein the expression amount of mRNA is measured by using at least one method selected from the group consisting of real-time PCR, gene expression profiling, and microarray analysis.

28. (canceled)

29. The method according to claim 26, wherein the expression amount of protein is measured by using at least one method selected from the group consisting of immunohistochemistry, immunofluorescence, mass spectrometry, flow cytometry, and Western Blotting.

30. A pharmaceutical composition comprising one or more DNA methyl transferase inhibitors for use in the treatment of a blood cancer patient identified or selected based on the expression amount of DUSP5.

31. (canceled)

32. A pharmaceutical composition comprising one or more DNA methyl transferase inhibitors for use in a blood cancer patient to whom the therapeutic effect of a drug is predicted by the method according to claim 2.

33. The pharmaceutical composition according to claim 30, wherein the DNA methyl transferase inhibitor is OR21, decitabine, azacytidine, RG-108, thioguanine, zebularine, SGI-110, SGI-1027, lomeguatrib, or procainamide hydrochloride.

34. (canceled)

35. The pharmaceutical composition according to claim 30, wherein the pharmaceutical composition further comprises one or more histone methyl transferase inhibitors.

36. The pharmaceutical composition according to claim 35, wherein the histone methyl transferase inhibitor is EPZ-6438, DS-3201b, GSK-126, Chaetocin, or BIX-01294.

37. A kit for identifying or selecting a blood cancer patient who may benefit from the treatment using a drug comprising one or more DNA methyl transferase inhibitors or a drug further comprising one or more histone methyl transferase inhibitors using the method according to claim 1, the kit comprising a reagent for measuring the expression amount of DUSP5 mRNA or DUSP5 protein in a sample obtained from the patient.

38. (canceled)

39. The kit for predicting the therapeutic effect in a blood cancer patient of a drug comprising one or more DNA methyl transferase inhibitors or a drug further comprising one or more histone methyl transferase inhibitors using the method according to claim 2, the kit comprising a reagent for measuring the expression amount of DUSP5 mRNA or DUSP5 protein in a sample obtained from the patient.

40. (canceled)
